(19) United States
(12) Patent Application Publication
(10) Pub. No.: US 2018/0374388 A1
(43) Pub. Date: Dec. 27, 2018

(54) SYSTEM AND METHOD FOR DISPLAYING DISCHARGE INSTRUCTIONS FOR A PATIENT

(71) Applicant: Cerner Innovation, Inc., Kansas City, KS (US)

(72) Inventors: David L. Compton, Lenexa, KS (US); Manikandan Nair, Olathe, KS (US); Frederick W. Eckertson, Kansas City, MO (US); Matthew D. Totten, Kansas City, MO (US); Mark B. Milligan, Kansas City, MO (US); James D. Eaton, Gardner, KS (US)

(21) Appl. No.: 15/959,558

(22) Filed: Apr. 23, 2018

Related U.S. Application Data
(63) Continuation of application No. 11/465,856, filed on Aug. 21, 2006, now abandoned.

(51) Int. Cl. G09B 23/28; G16H 15/00; G16H 10/60

(52) U.S. Cl. CPC G09B 23/28; G16H 15/00; G16H 10/60; G06Q 50/24; G06F 19/3481

(57) ABSTRACT

A computer system and method for displaying suggested discharge instructions for a patient is provided. Patient data and discharge qualifiers are accessed. The patient data and the discharge qualifiers are utilized to determine suggested discharge instructions for the patient, where the suggested discharge instructions comprise patient education instructions, follow-up care instructions, follow-up appointment instructions and prescription instructions for the patient. The suggested discharge instructions for the patient are displayed substantially simultaneously.

### CROSS-REFERENCE TO RELATED APPLICATIONS

This application is a continuation of U.S. application Ser. No. 11/465,856, filed Aug. 21, 2006, and entitled “SYSTEM AND METHOD FOR DISPLAYING DISCHARGE INSTRUCTIONS FOR A PATIENT,” the entire contents of which are incorporated herein by reference in their entirety.

### STATEMENT REGARDING FEDERALLY SPONSORED RESEARCH OR DEVELOPMENT

Not applicable.

### BACKGROUND

Upon discharge from a hospital, physicians provide a patient with discharge instructions. In particular, a physician may provide the patient with patient education instructions, follow-up care information, follow-up appointments and prescriptions.

Currently, a physician must manually write down the discharge instructions or manually input patient education instructions, follow-up care information, follow-up appointments and prescriptions into a computerized system. Not only is this method of discharging a patient inefficient, these administrative tasks ultimately lead to patient dissatisfaction. Instead of delivering patient care and reviewing results, physicians are busy manually entering information needed for patient discharge.

While some healthcare information systems offer a mechanism for a healthcare provider to manually select and input different discharge instructions, no system automatically and simultaneously provides multiple recommended discharge instructions specific to the patient information.

### SUMMARY

In one embodiment, a method in a computerized healthcare environment for displaying discharge instructions for a patient is provided. Patient data and discharge qualifiers are accessed. The patient data and the discharge qualifiers are utilized to determine suggested discharge instructions for the patient, where the suggested discharge instructions comprise patient education instructions, follow-up care instructions, follow-up appointment instructions and prescription instructions for the patient. The suggested discharge instructions for the patient are displayed substantially simultaneously.

In another embodiment, a user interface for displaying discharge instructions for a patient is provided. The user interface in comprises a first display area configured to display suggested patient education instructions for a patient; a second display area configured to display suggested follow-up care instructions for the patient; a third display area configured to display suggested follow-up appointment instructions for the patient; and a fourth display area configured to display suggested prescriptions instructions for the patient.

In yet another embodiment, a computer system for displaying discharge instructions for a patient is provided. The system comprises a first accessing component for accessing patient data and a second accessing component for accessing discharge qualifiers. The system further comprises a utilizing component for utilizing the patient data and the discharge qualifiers to determine suggested discharge instructions for the patient, wherein the discharge instructions comprise patient education instructions, follow-up care instructions, follow-up appointment instructions and prescription instructions for the patient. A displaying component is provided for displaying the suggested discharge instructions for the patient substantially simultaneously.

### BRIEF DESCRIPTION OF THE DRAWINGS

The present invention is described in detail below with reference to the attached drawing figures, wherein:

FIG. 1 is a block diagram of a computing system environment suitable for use in embodiments of the present invention;

FIG. 2 is a block diagram of an exemplary system for use in implementing embodiments of the present invention;

FIG. 3 is a flow diagram of a method for displaying suggested discharge instructions for the patient in accordance with an embodiment of the present invention;

FIG. 4 is an exemplary user interface for displaying a list of patients awaiting discharge in accordance with an embodiment of the present invention;

FIG. 5. is an exemplary user interface displaying suggested discharge instructions for a patient in accordance with an embodiment of the present invention;

FIG. 6 is an exemplary user interface displaying a prescription instruction change documentation form in accordance with an embodiment of the present invention;

FIG. 7 is an exemplary user interface displaying patient education instruction change documentation form in accordance with an embodiment of the present invention;

FIG. 8 is an exemplary user interface displaying a follow-up appointment instruction change documentation form in accordance with an embodiment of the present invention;

FIG. 9A is an exemplary user interface displaying a follow-up appointment instruction change documentation form in accordance with an embodiment of the present invention;

FIG. 9B is an exemplary user interface displaying a follow-up appointment instruction change documentation form in accordance with an embodiment of the present invention; and

FIG. 10 is an exemplary user interface displaying a suggested discharge for a patient in accordance with an embodiment of the present invention.

### DETAILED DESCRIPTION OF THE INVENTION

Embodiments of the present invention are directed to systems and methods in a healthcare environment for displaying discharge instructions for a patient. The systems and methods of the present invention not only have the ability to suggest discharge instructions for a patient, but display the different discharge instructions simultaneously and automatically. The system and method allows a user to modify, add or delete the suggested discharge instructions.

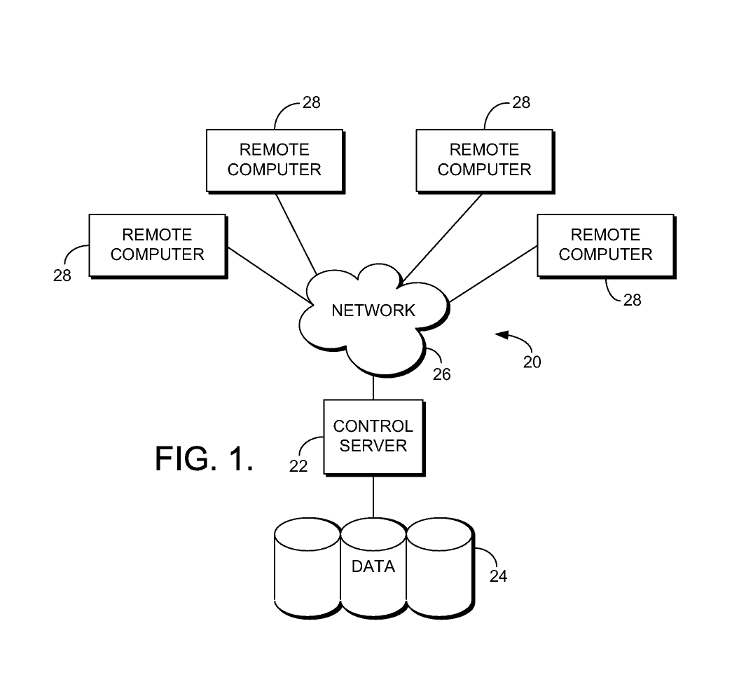

Having briefly described an overview of the present invention, embodiments of the invention will be discussed with reference to FIGS. 1-10.

With reference to FIG. 1, an exemplary medical information system for implementing the invention includes a general purpose-computing device in the form of server 22. Components of server 22 may include, but are not limited to, a processing unit, internal system memory, and a suitable system bus for coupling various system components, including database cluster 24 to the control server 22. The system bus may be any of several types of bus structures, including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Server 22 typically includes therein or has access to a variety of computer readable media, for instance, database cluster 24. Computer readable media can be any available media that can be accessed by server 22, and includes both volatile and nonvolatile media, removable and non-removable media. By way of example, and not limitation, computer readable media may comprise computer storage media and communication media. Computer storage media includes volatile and nonvolatile, removable and non-removable media implemented in any method or technology for storage of information, such as computer readable instructions, data structures, program modules or other data. Computer storage media includes, but is not limited to, RAM, ROM, EEPROM, flash memory or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by server 22. Communication media typically embodies computer readable instructions, data structures, program modules, or other data in a modulated data signal, such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media, such as a wired network or direct-wired connection, and wireless media, such as acoustic, RF, infrared and other wireless media. Combinations of any of the above should also be included within the scope of computer readable media.

The computer storage media, including database cluster 24, discussed above and illustrated in FIG. 1, provide storage of computer readable instructions, data structures, program modules, and other data for server 22.

Server 22 may operate in a computer network 26 using logical connections to one or more remote computers 28. Remote computers 28 can be located at a variety of locations in a medical or research environment, for example, but not limited to, clinical laboratories, hospitals, other inpatient settings, a clinician's office, ambulatory settings, medical billing and financial offices, hospital administration, veterinary environment and home healthcare environment. Clinicians include, but are not limited to, the treating physician, specialists, such as surgeons, radiologists and cardiologists, emergency medical technologists, physician's assistants, nurse practitioners, nurses, nurse's aides, pharmacists, dieticians, microbiologists, laboratory experts, genetic counselors, researchers, veterinarians and the like. The remote computers may also be physically located in non-traditional medical care environments so that the entire healthcare community is capable of integration on the network.

Remote computers 28 may be a personal computer, server, router, a network PC, a peer device, other common network node or the like, and may include some or all of the elements described above relative to server 22. Computer network 26 may be a local area network (LAN) and/or a wide area network (WAN), but may also include other networks. Such networking environments are commonplace in offices, enterprise-wide computer networks, intranets and the Internet. When utilized in a WAN networking environment, server 22 may include a modem or other means for establishing communications over the WAN, such as the Internet. In a networked environment, program modules or portions thereof may be stored in server 22, or database cluster 24, or on any of the remote computers 28. For example, and not limitation, various application programs may reside on the memory associated with any one or all of remote computers 28. It will be appreciated that the network connections shown are exemplary and other means of establishing a communications link between the computers may be used.

A user may enter commands and information into server 22 or convey the commands and information to the server 22 via remote computers 28 through input devices, such as keyboards, pointing devices, commonly referred to as a mouse, trackball, or touch pad. Other input devices may include a microphone, satellite dish, scanner, or the like. Server 22 and/or remote computers 28 may have any sort of display device, for instance, a monitor. In addition to a monitor, server 22 and/or computers 28 may also include other peripheral output devices, such as speakers and printers.

Although many other internal components of server 22 and computers 28 are not shown, those of ordinary skill in the art will appreciate that such components and their interconnection are well known. Accordingly, additional details concerning the internal construction of server 22 and computer 28 need not be disclosed in connection with the present invention.

Although the method and system are described as being implemented in a WINDOWS operating system operating in conjunction with an Internet-based system, one skilled in the art would recognize that the method and system can be implemented in any system.

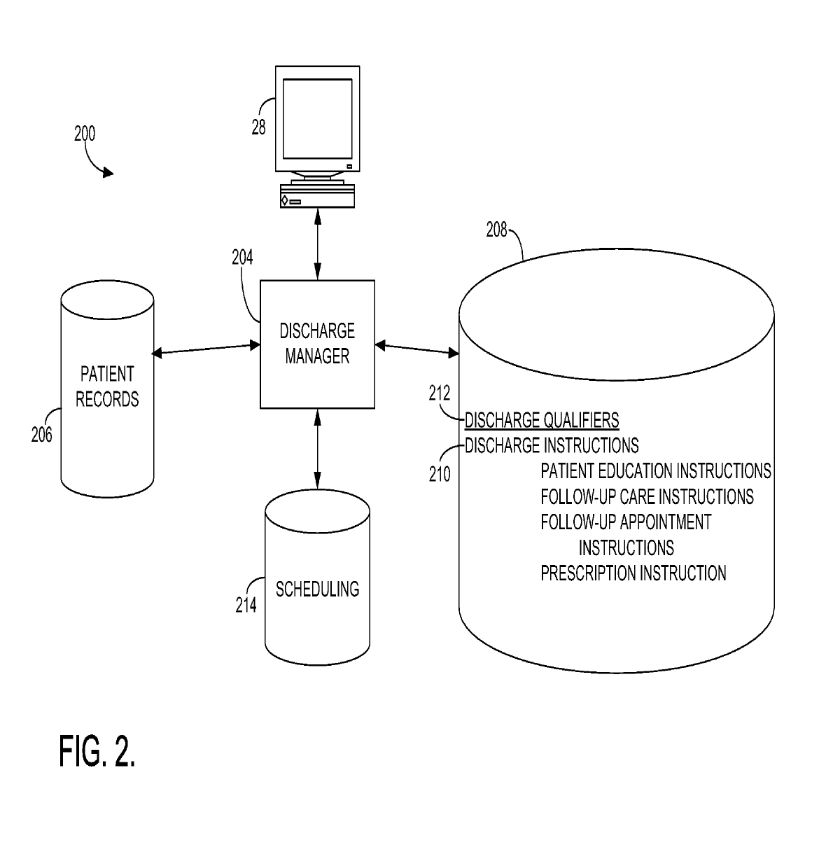

Referring next to FIG. 2, a block diagram of an exemplary system for use in implementing one or more embodiments of the present invention is shown. A system 200 comprises a discharge manager 204, which is in communication with patient records 206, a discharge database 208 and a scheduling database 214.

Discharge manager 204 may be in communication with or located on a remote computer 28 to be used by a user. The user may be a healthcare provider, such as a nurse, doctor or other healthcare worker. Discharge manager 204 is in communication with patient records 206, discharge database 208 which may include discharge qualifiers 212 and suggested discharge instructions 210, and a scheduling database 214. It will be appreciated that patient records 206, discharge instructions 210, discharge qualifiers 212 and a scheduling database 214 may also be stored in a common database or multiple databases.

Patient records 206 may include patient data, such as patient demographic information, address, age, gender, weight, race, recorded problems or diagnoses, procedures performed, results, orders, tasks, insurance provider and a variety of other patient information. In one embodiment, patient records are stored in a patient's electronic medical record (EMR).

Discharge database 208 contains discharge qualifiers 212 and discharge instructions 210. The discharge manager 204 accesses discharge qualifiers 212 and compares them to patient data accessed from patient records 206. Discharge qualifiers 212 identify what patient data qualifies a patient to receive suggested discharge instructions 210. The suggested discharge instructions may be displayed to a user, such as an emergency room doctor or nurse, via computer 28. The discharge qualifiers 212 may be a variety of information including one or more diagnosis codes (such as primary and secondary diagnosis codes), patient demographic information (e.g. patient is a smoker, patient is overweight), patient complaints and a variety of other patient data. The discharge qualifiers 212 may be associated with suggested discharge instructions 210. The suggested discharge instructions 212 may include patient education instructions, follow-up care instructions, follow-up appointment instructions and prescription instructions. Table 1, below, shows a discharge qualifier 212, in the form of a discharge diagnosis code, associated with suggested discharge instructions 210.

Patient education instructions comprise identification of one or more patient education references, articles and literature relating to healthcare and the patient. Follow-up care instructions comprise identification of a time-period for follow-up by the patient and name and contact information of a suggested healthcare provider. Follow-up appointment instructions may comprise scheduled follow-up appointments for the patient, including place, time and date of the follow-up appointment. Prescription instructions may comprise suggested drugs or treatment for the patient, dosage amount, time and instructions, possible contraindications, side-effects, generic equivalents and other prescription information.

TABLE 1

| Qualifier | Suggested Discharge Instructions |
|---|---|
| Diagnosis Code 786.5 | Follow-Up Care Instruction: Seek follow-up |
| (Chest Pain) | appointment in 3-5 days of discharge date |
| | Patient Education Instruction: Maintain low |
| | salt diet, provide chest pain prevention print- |
| | out |
| | Prescription Instruction: 1 patch per day of |
| | Nitroglycerin |
| | Follow-up Appointment Instruction: Access |
| | scheduling application to schedule |
| | appointment close to home with 5 days of |
| | discharge date |

As shown in FIG. 2, the discharge manager 204 is capable of communicating with patient records 206, suggested discharge instructions 210 and discharge qualifiers 212, for example, for the suggested discharge instructions for a patient. Patient data from patient records 206 are compared with the discharge qualifiers 212 to suggest appropriate discharge instructions 210 for the patient.

By way of example and not by limitation, the discharge manager 204 accesses patient records 206 and determines that a patient has chest pain diagnosis code 786.5 as shown in Table 1. The discharge manager accesses discharge qualifiers 212 and determines that patients with chest pain diagnosis code 786.5 qualify for suggested patient education instructions, follow-up care instructions, follow-up appointment instructions and prescription instructions 210.

For example, if the patient has chest pain diagnosis code 786.5 it is determined that the patient qualifies for suggested patient education instructions for maintaining a low salt diet and prevent chest pain. Furthermore, chest pain diagnosis code 786.5 qualifies the patient for chest pain suggested follow-up care discharge instructions. It is determined that the patient should be provided with suggested follow-up care instructions which indicate that the patient should follow up with a physician in 3-5 days.

Furthermore, a patient that has chest pain diagnosis code 786.5 qualifies for suggested follow-up appointment instructions. For instance with reference to Table 1, suggested discharge follow-up appointment instructions provide a date and time to meet with a physician (e.g. 11:00 A.M. on 12/05/2006). A scheduling application 214 may be accessed to determine an appropriate date, time and location for a follow-up appointment. A chest pain diagnosis code 786.5 qualifies the patient for suggested prescription instructions at discharge. It is determined that the patient with a diagnosis code of 786.5 should be provided with suggested patient discharge prescription instructions including the prescription and dosage amount to treat the heart condition (e.g. 1-Patch per day of Nitroglycerin).

In an alternative example, the discharge qualifier 212 includes data identifying the patient as a smoker. The discharge manager 204 accesses patient records 206 and determines that a patient is a smoker. The discharge manager 204 accesses discharge qualifiers 212 and determines that being a smoker qualifies a patient for suggested patient education discharge instructions 210, such as smoking cessation education.

In addition, communication between the discharge manager 204 and the patient records 206 discharge qualifiers 212, suggested discharge instructions 210, scheduling application 214, and remote computer 28 may be via one or more networks, which may comprise one or more wide area networks (WANs) and one or more local area networks (LANs), as well as one or more public networks, such as the Internet, and one or more private networks.

The discharge manager 204 may be accessed in a variety of ways within the scope of the present invention. For example, in some embodiments, an entity may have a native clinical computing system, which may be able to communicate with the discharge manager 204. In other embodiments, a client application associated with the discharge manager 204 may reside on an entity's computing device facilitating communication with the discharge manager 204. In further embodiments, communication may simply be a web-based communication, using, for example, a web browser to access the discharge manager 204 via the Internet. Any and all such variations are contemplated to be within the scope of embodiments of the present invention. In one embodiment, unified healthcare architecture, such as Cerner Millennium® by Cerner Corporation of Kansas City, Mo. may be utilized.

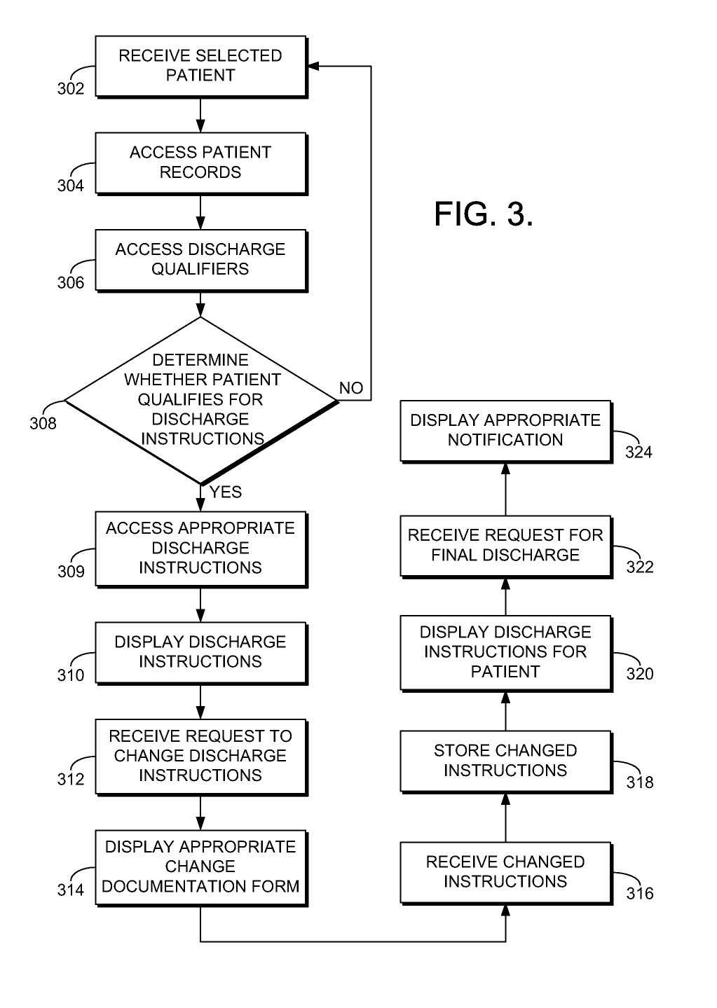

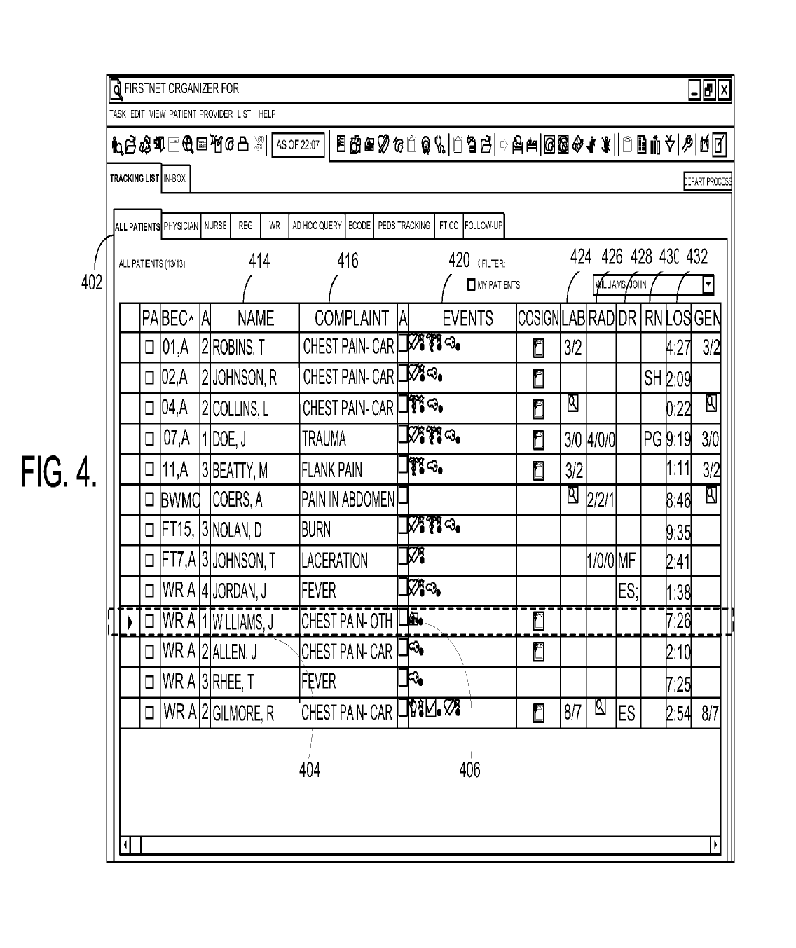

Referring to FIG. 3, a method for displaying suggested discharge instructions for a patient is shown. At step 302, the appropriate patient is identified. A patient may be identified, by scanning a scanned barcode or a user selecting a patient from a patient list. For example, with reference to FIG. 4, a clinician may select a patient from patient list 402. Patient list 402 is a list of patients being treated in the emergency room and may include information regarding the patients, such as the patient's name 414, complaint 416, events 420, laboratory events 424, radiology events 426, the physician's initials 428, assigned nurse 430 and length of stay 432.

The method for displaying discharge instructions can begin with a single action or one touch of a user. For example, a user, such as an emergency room healthcare provider, may select a patient from a list to trigger the collection and display suggested discharge instructions. For example, with reference to FIG. 4, selection of patient Williams 404 from the patient discharge list 402, will automatically trigger the collection and display of suggested discharge instructions. Alternatively, in the context of a single patient, from the patient's electronic record, a user may select a “discharge” option to trigger collection and display of the suggested discharge instructions. It will be appreciated that the single action or one touch of a user may be a variety of single actions or one touches of user.

Referring again to FIG. 3, at step 304, the patient's records are accessed. In one embodiment, the patient records may include patient data, such as the patient demographic information, patient address, age, gender, weight, race, tasks and orders for the patient, recorded problems or diagnoses, procedures performed, insurance provider and a variety of other patient data. In one embodiment, patient records are maintained in an electronic medical record.

At step 306, discharge qualifiers are accessed. A list of qualifiers that qualify a patient for specific discharge instructions may be stored in a database or table. At step 308, it is determined whether the patient qualifies for one or more discharge instructions. For example, the patient records may indicate that the patient has a diagnosis code of 786.5. Table 1, shown above, may be accessed. The diagnosis code 786.5 as shown in Table 1, above, qualifies the patient to receive suggested discharge instructions. At step 309, the appropriate discharge instructions are accessed for the patient. It will be appreciated that a discharge qualifier may qualify the patient for one discharge instruction or for multiple discharge instructions.

Once the suggested discharge instructions for the selected patient have been determined, the suggested discharge instructions are displayed at step 310. In one embodiment, suggested patient education instructions, follow-up care instructions, follow-up appointment instructions and prescription instructions are displayed. In another embodiment, at least two of suggested patient education instructions, follow-up care instructions, follow-up appointment instructions and prescription instructions are displayed.

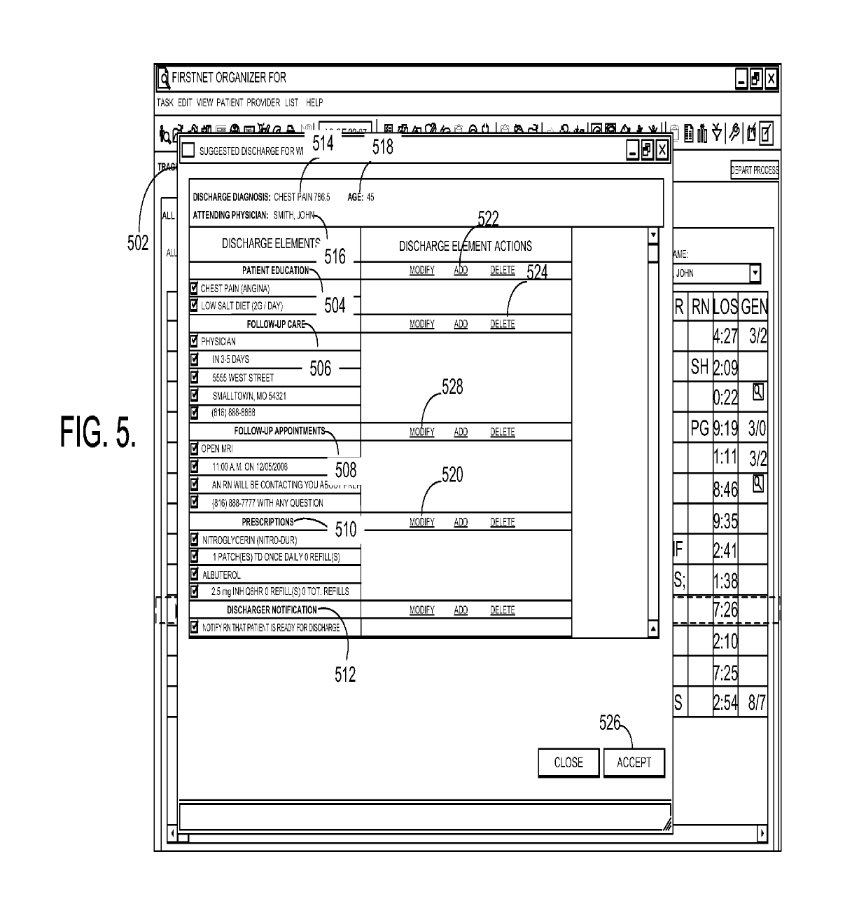

With reference to FIG. 5, an exemplary graphical user interface 502 displaying suggested discharge instructions for the patient is shown. The user interface 502 for the patient may include patient information, such as the name of the patient and a discharge diagnosis code 514, the name of the attending physician 516 and the age of a selected patient 518. Also displayed are the suggested discharged instructions for the patient including suggested patient education instructions 504, follow-up care instructions 506, follow-up appointment instructions 508 and prescription instructions 510. In some instances, the suggested patient education instructions 504, follow-up care instructions 506, follow-up appointment instructions 508 and prescription instructions 510 for the patients are based upon to the patient's discharge diagnosis code 514. One of skill in the art will appreciate that any variety of codes or identifiers for discharge diagnosis codes may be utilized and that the suggested discharge instructions may be based upon a variety of patient qualifiers.

Patient education instructions may be related to a patient's diagnosis or any other health risks the patient may have. In one embodiment, if the patient is complaining of chest pains has a discharge diagnosis code 514 of “786.5.” It is determined that patient education instructions are appropriate for the patient and are suggested to the healthcare provider in the exemplary user interface 502. For example, patient education instructions 504 for “chest pain” include a description of what chest pain is, how to abort chest pain and ways to prevent chest pain, such as maintaining a low salt diet. If a healthcare provider wants to add additional patient education instructions or change the suggested patient education instructions 504, the suggested patient education instructions 504 may be modified as described in further detail below.

Follow-up care instructions 506 provide suggested follow-up time ranges for a patient to meet with a physician. A discharge diagnosis code of 786.5 qualifies the patient for suggested follow-up instructions 506. For example, if a patient has an emergency encounter on Mar. 1, 2006, the discharge manager 204 of FIG. 2 accesses patient records 206 and suggested discharge instructions 210 to determine the appropriate time frame for follow up with a physician for the patient. For example, the patient records indicate a diagnosis code 786.5 for chest pain and the discharge instructions 210 suggest patients with chest pain should seek follow up care in n+3 days, n+4 days or n+5 days, where N is the date of discharge. Thus, the range of “in 3-5 days” is displayed as a suggested follow-up care instruction for the patient.

In another embodiment, the suggested follow-up care instructions include actual dates, such as Mar. 4, 2006, Mar. 5, 2006 and Mar. 6, 2006, which have been determined using the time frame for the suggested follow-up care instructions 210 and the patient's anticipated discharge date accessed from patient records 206. In another embodiment, the discharge diagnosis manager 204 evaluates the other patient data from patient records 206, such as the patient's diagnosis, address, age, insurance provider and will suggest a local physician who specializes in the particular area needed, with that particular age of patient and/or who accepts the patient's insurance. Referring again to FIG. 5, the name, address and phone number of the suggested physician may be displayed in the suggested follow-up care instruction 506.

In one embodiment, the suggested follow-up appointment instructions 508 provide a scheduled appointment with a healthcare provided or for a particular procedure. The scheduled appointment is suggested according to patient data 206 and suggested discharge instructions 210 of FIG. 2. For example, the discharge manager 204 determines that the patient had an emergency encounter with a chest pain diagnosis code 786.5 on Dec. 1, 2006 from patient records 206. The suggested discharge instructions 210 indicate the patient should follow up with an MRI within five days. The discharge manager 204 accesses a scheduling application 214 with scheduling capabilities for MRI appointments and determines an appointment is available on Dec. 5, 2006. The discharge manager 204 provides a suggested discharge follow-up appointment at 11:00 A.M. on Dec. 5, 2006 at a local MRI office as the suggested follow-up appointment instruction 508. In another embodiment, the discharge manager 204 suggests that this patient be contacted by a nurse regarding a scheduled appointment and provides a number for the patient to call if the patient has any questions. If the suggested follow up appointment date or time needs to be changed, the user can schedule an appointment that will more appropriately suit the needs of the patient as discussed in greater detail below.

Referring again to FIG. 5, suggested prescription instructions 510 provide a suggested prescription along with a suggested dosage. The discharge manager 204 of FIG. 2 accesses patient records 206 for patient data such as the patient's age, weight and medical history and discharge diagnosis code. The accessed patient data is utilized to determine the appropriate prescription and dosage. For example, the suggested discharge prescription 510 for a 45 year old male patient with a chest pain diagnosis code 786.5 is one (1) patch Nitroglycerin once daily and 25 mg Albuterol inhalation every six (6) hours. The discharge manager 204 may access a prescription application to help determine the appropriate prescription. A prescription application, such as Easy Script by Cerner Corporation of Kansas City, Mo. may be used. In another embodiment, the system notifies a physician, via an indicator, if an alert is associated with the suggested prescription. For instance, based on patient data, a patient may be allergic to the suggested prescription or may be taking another medication that cannot be combined with the suggested prescription.

Referring again to FIG. 3, at step 312, a request to change discharge instructions is received. The request to change may be to modify the suggested discharge instructions, add suggested discharge instructions or delete suggested discharge instructions. With reference to FIG. 5, these three change options are represented by the fields modify 520, add 522 and delete 524. Each category of suggested discharge instructions may be modified. For example, if a user wants to modify the suggested prescription instructions 510, the user will select the option modify 520 to modify the suggested prescription instructions 510.

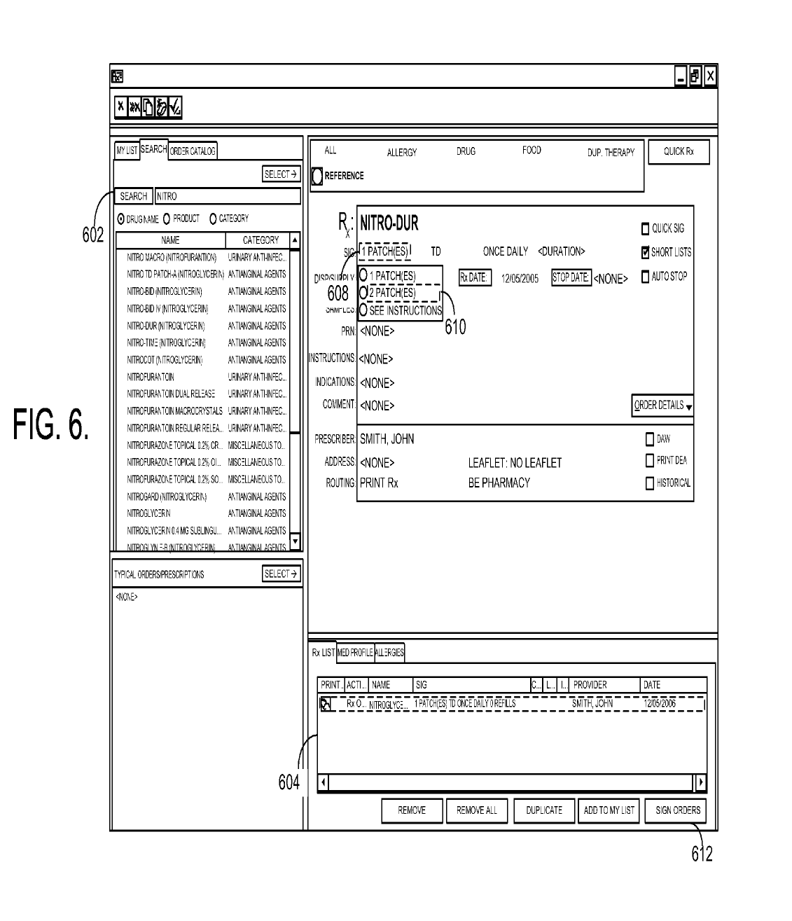

Referring to FIG. 3, at step 314, the appropriate change documentation form for the selected modification is displayed. For example with reference again to FIG. 5, the suggested discharge prescription instructions 510 are for one (1) patch per day of Nitroglycerin. The user selects modify 520 to change the prescription instructions 510. With reference to FIG. 6, the appropriate prescription documentation form 602 is displayed. A prescription list 604 is displayed detailing the suggested medication prescribed and the dosage that is recommended. In one embodiment a user may change the dosage from one (1) patch 608 per day to two (2) patches 610 per day. When the user has completed making changes to the suggested prescription instruction, the user selects sign orders 612 to change the suggested prescription instruction.

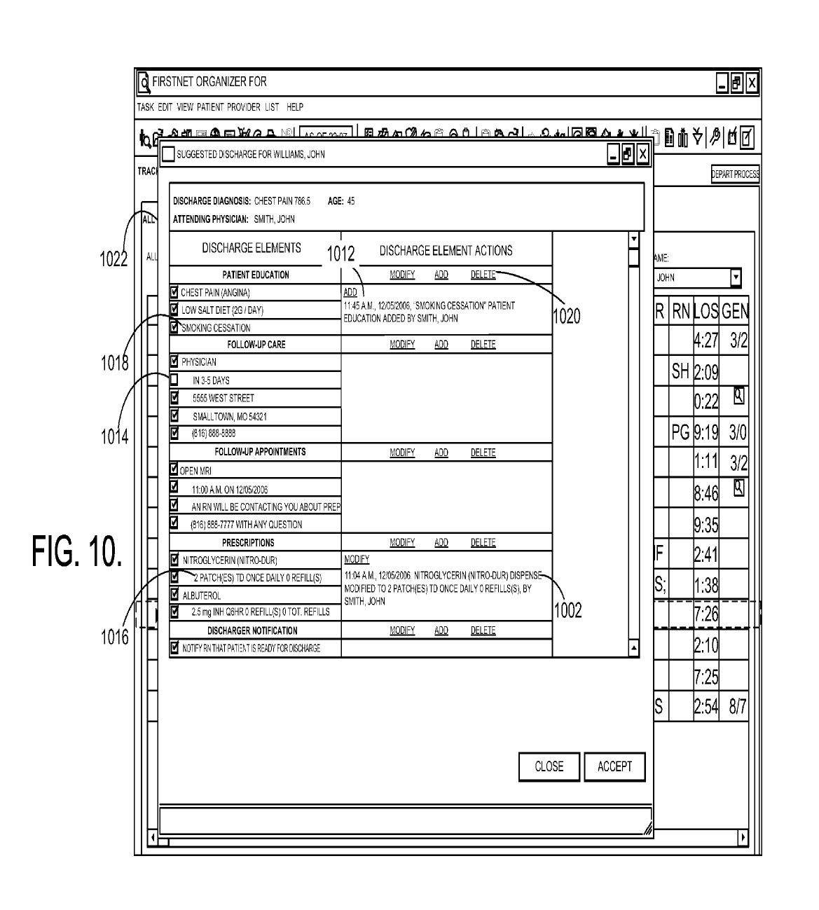

Referring now to FIG. 10, the newly modified dosage of two (2) patches 1016 of Nitroglycerin is displayed on user interface 1022. Information as to the type of changes (modify, add or delete) made, the time and date of the change, the name of the physician or healthcare provided making the change and content of the information that was changed are displayed at 1002. In another embodiment, the newly modified information will be highlighted in a different color or font indicating to the user that changes have been made and these changes are now incorporated into the suggested discharge instructions for the patient. In another embodiment, a symbol or identifier is displayed to indicate the newly modified information.

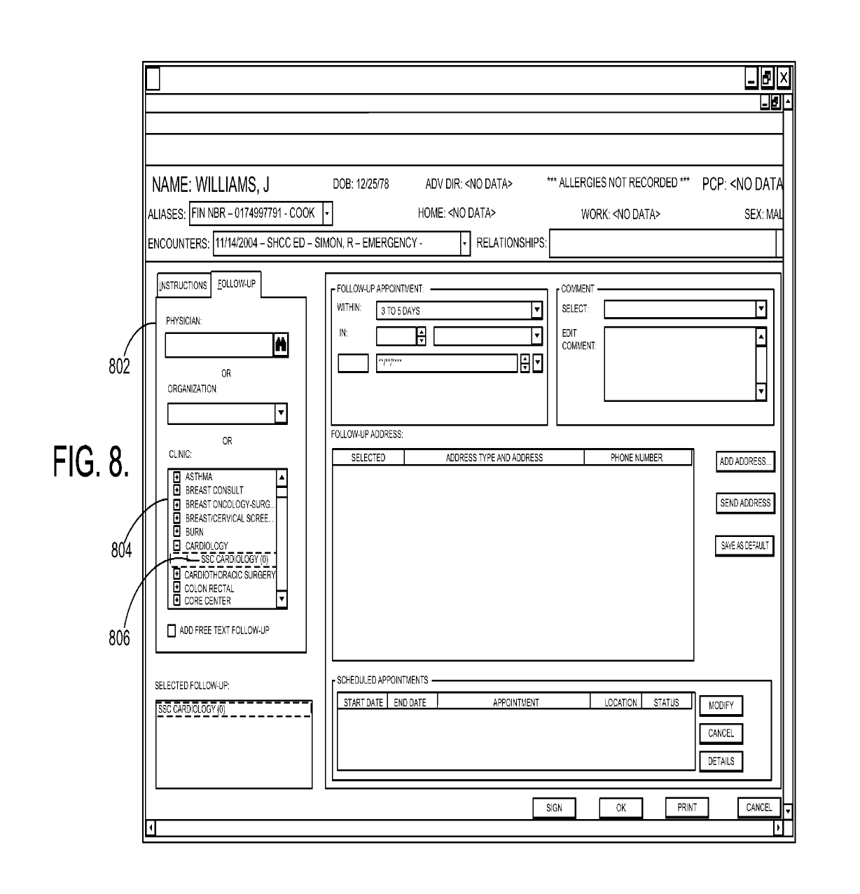

Referring again to FIG. 5, modifications may be made to the suggested follow-up appointment instructions 508. To make a modification, the user selects modify 528 to display the appropriate follow-up appointment documentation form. An exemplary appointment documentation form 802 is shown in FIG. 8. A scheduling application, such as Scheduling Management by Cerner Corporation of Kansas City, Mo. is accessed to schedule follow-up appointments for the patient. For example, if a healthcare provider wants to schedule an appointment for the patient in the SSC Cardiology Clinic, the healthcare provider selects desired SSC clinic 806 within the clinic display window 804.

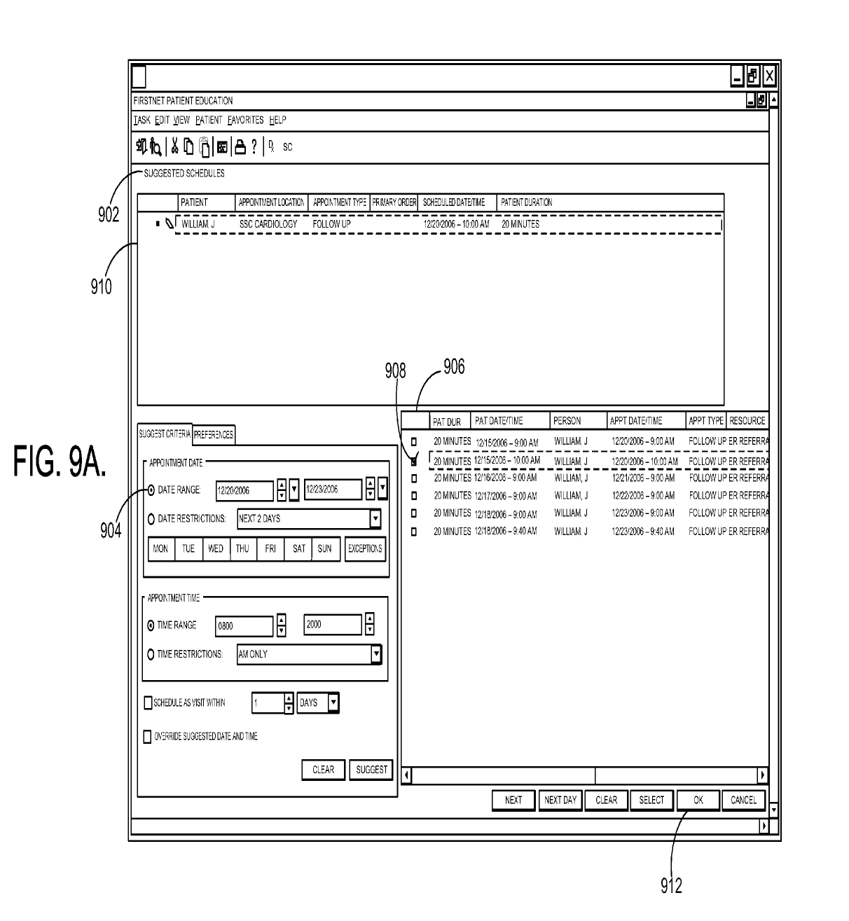

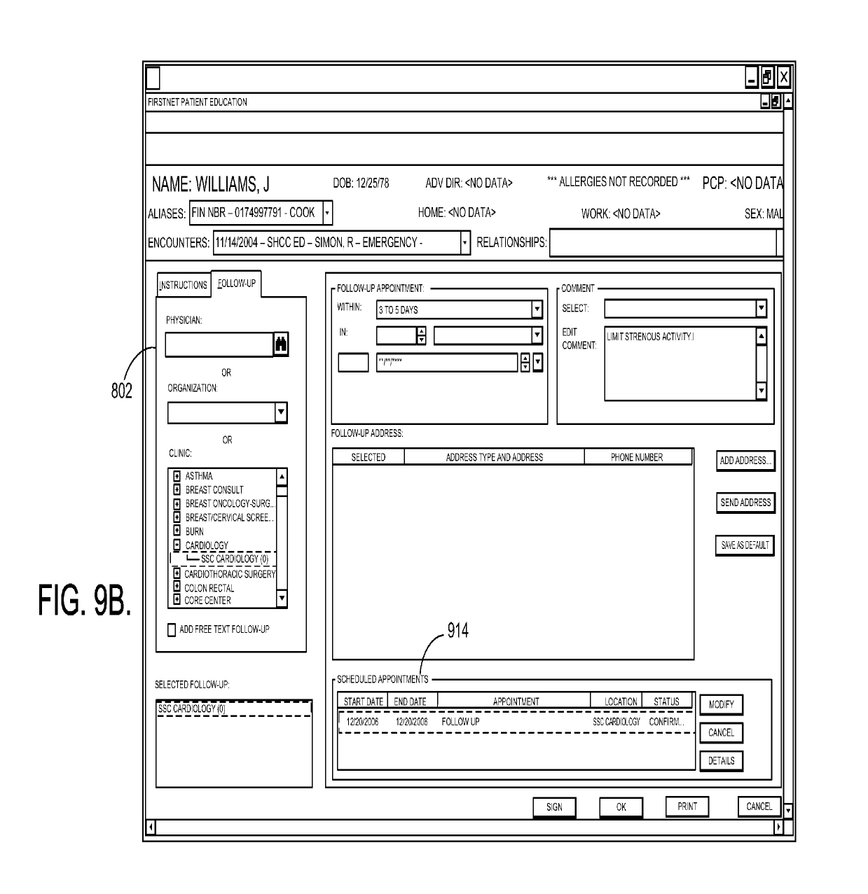

When the user has completed selecting the desired clinic or physician, with reference to FIG. 9A, a suggested schedule documentation form 902 is launched. In one embodiment, within the suggested schedule documentation form 902, a date range 904 for possible appointments is suggested and a list of available times and dates of potential appointments according to the specified date range is displayed. In one embodiment, when a user has selected an appointment from the available appointments window 906, the selection is automatically placed onto the suggested schedule window 910.

Referring now to FIG. 9B, once the user has selected the desired appointment date and time, the follow-up appointment documentation form 802 is automatically updated. The newly scheduled appointment is displayed as a scheduled appointment 914. Once the scheduled appointment is verified the suggested follow-up appointment discharge instructions for the patient are updated to include the newly scheduled appointment.

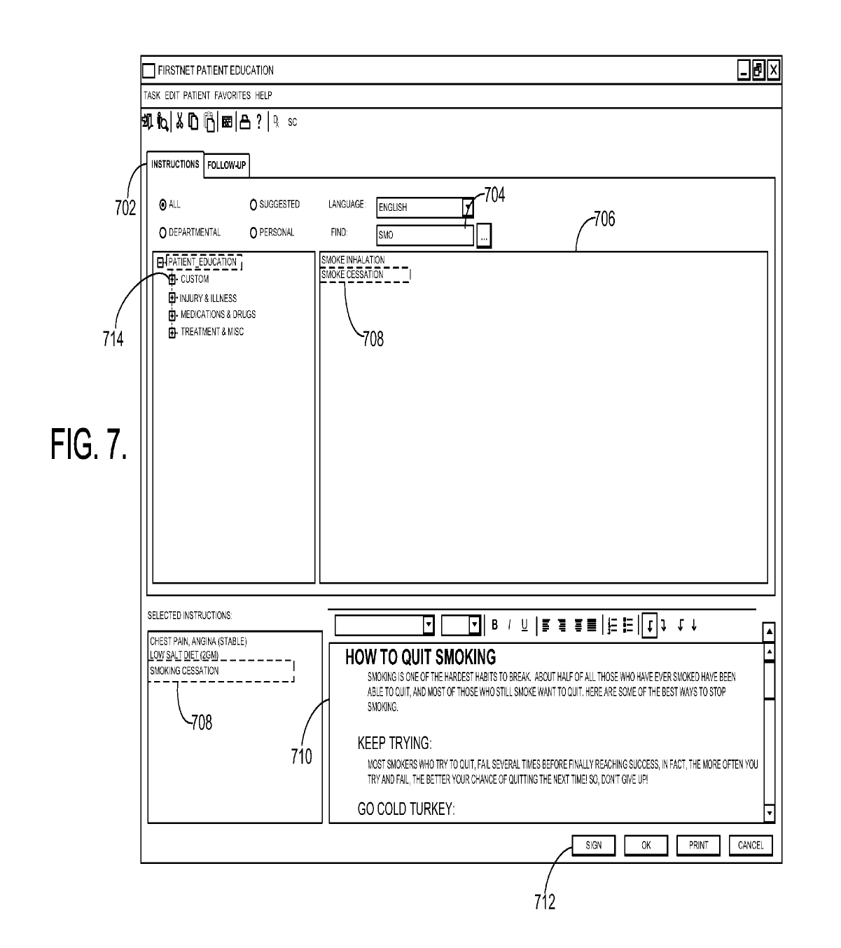

Referring again to FIG. 5, a healthcare provider may add discharge instructions beyond the suggested discharge instructions. For example, if a patient is a smoker, the healthcare provider may add suggested patient education instructions 504 to provide the patient with literature on how to quit smoking. In one embodiment, selecting add 522 of FIG. 5 launches a patient education change documentation form 702 as shown in FIG. 7. The user may search for patient education instructions in search field 704 and select a topic from the search results 706. After the topic 708 is selected from the search results 706, content for the selected result is automatically displayed in a content display area 710.

In another embodiment, if a user wishes to generate their own education comments, the user can select custom 714 and input education instructions in the content display area 710. The user has the option of incorporating the input education instructions and designating the instructions into the database as personal or public education instructions. Public education instructions may be accessed by anyone who has authority to enter the system. Personal instructions are accessible only to the user who input the instructions.

Once the addition of information is complete, the user selects sign 712 to include the modified information in the suggested patient education instructions. The newly added smoking cessation suggested patient education discharge instructions can be seen at 1018 of FIG. 10. The type of change, the time and date of the change and the name of the physician who made the change are displayed at 1012.

Referring again to FIG. 5, a user has the option to delete discharge instructions from the suggested discharge instructions provided. In one embodiment, all discharge instructions are pre-selected, therefore, if a user wishes to delete instructions, the user unchecks the unwanted instructions. If a user wants to delete “IN 3-5 DAYS” under the suggested follow-up care instructions 506, the user unchecks the box located next to the unwanted instructions. With reference to FIG. 10, unchecked box 1014 indicates to the user that the specific instructions, “IN 3-5 DAYS,” will not be included in the suggested discharge instructions.

Alternatively, the user may select delete 524 to delete unwanted instructions. The selection of delete 524 will uncheck the unwanted discharge instructions and in some instances will also erase the type of change (modify, add or delete) was displayed, such as the time and date of the change, the name of the physician who made the change and what information was changed. For example, with reference to FIG. 10, the information provided at 1012 and 1002 is erased if delete 1020 is selected. A warning may be displayed informing the user that information is about to be lost. Alternatively, the selection of delete 1020 unchecks all of the newly added or modified information without erasing the information. For example, with reference to FIG. 10, if a user selects delete 1020, smoking cessation 1018 is automatically unchecked, but the information at 1012 is preserved.

Referring again to FIG. 3, at step 322, a request for a final discharge is received. For example, once a user is satisfied with the content of the suggested discharge instructions, the user may request the final discharge of a patient. For example with reference to FIG. 5, a user may select 512 to notify that the patient is ready for discharge. In one embodiment, once a patient is finally discharged, the follow-up physicians selected will receive a fax of the clinical summary including the suggested discharge instructions. In another embodiment, clinical and patient summaries including the suggested discharge instructions are printed and the information is stored in the patient's EMR.

At step 324, appropriate notification of the patient's discharge is displayed. With reference to FIG. 4, in one embodiment, the notification is a house symbol 406. However, one with ordinary skill in the art will appreciate that many symbols and indications exist that may provide a user a notification that the patient has been finally discharged. Alternatively, the patient's name may be added to a final discharge queue.

The present invention has been described in relation to particular embodiments, which are intended in all respects to illustrate rather than restrict. Alternative embodiments will become apparent to those skilled in the art that do not depart from its scope. Many alternative embodiments exist, but are not included because of the nature of the invention. A skilled programmer may develop means for implementing the aforementioned improvements without departing from the scope of the present invention.

It will be understood that certain features and sub-combinations of utility may be employed without reference to features and sub-combinations and are contemplated within the scope of the claims. Furthermore, the steps performed need not be performed in the order described.

The invention claimed is:

1. One or more computer storage media storing computer-executable instructions embodied thereon that, when executed by a computing device, perform a method for displaying discharge instructions for a patient, the method comprising:
receiving a single action of a user to display discharge instructions for a patient; and in response to the single action, automatically:
(a) accessing, by a discharge manager in operable communication with a control server, patient data;
(b) accessing a plurality of non-patient-specific discharge qualifiers, each discharge qualifier identifying a diagnosis or symptom description and a demographic trait, and each discharge qualifier associated with one or more suggested discharge instructions based on the discharge qualifier;
(c) applying, by the discharge manager, the plurality of discharge qualifiers to the patient data;
(d]) determining, based on the applying, at least one discharge qualification identifier for the patient, wherein the at least one discharge qualification identifier is generated if the patient data includes one or more discharge qualifiers applied from the accessed plurality of discharge qualifiers;
(e) generating, by the discharge manager, a set of discharge instructions for the patient based on the one or more discharge qualifiers associated with the at least one discharge qualification identifier; and
(f) simultaneously displaying the set of discharge instructions associated with the patient including each of:
patient education instructions, follow-up care instructions, follow-up appointment instructions, and prescription instructions for the patient, where the prescription instructions for the patient are automatically suggested along with a suggested dosage based on patient information in an electronic medical record associated with the patient.

2. The media of claim 1, wherein each discharge qualifier includes a diagnosis code.

3. The media of claim 1, wherein the patient education instructions comprise identification of one or more patient education references appropriate for the patient at discharge.

4. The media of claim 1, wherein the prescription instructions comprise identification of prescriptions for the patient at discharge, wherein the prescription instructions are determined utilizing a pharmacy application.

5. The media of claim 1, wherein the follow-up care instructions comprise identification of a time period for follow-up care for the patient after discharge.

6. The media of claim 1, wherein the follow-up appointment instructions comprise one or more scheduled follow-up appointments for a patient after discharge, wherein the follow-up appointment instructions are determined using a scheduling application.

7. The media of claim 1, further comprising:
receiving a request to change one or more of the suggested discharge instructions for the patient, wherein the request to change is a request to modify, request to add, or a request to delete.

8. The media of claim 7, further comprising:
receiving changes to one or more of the suggested discharge instructions for the patient;
storing the changes to one or more of the suggested discharge instructions in the patient's electronic medical record; and
displaying the changes to the one or more suggested discharge instructions.

9. The media of claim 1, wherein the single action is a user selecting a patient from a list of patients to be discharged.

10. The media of claim 1, wherein the single action is a user selecting to discharge a patient from the patient's electronic medical record.

11. One or more computer hardware storage media storing computer-executable instructions for presenting a user interface for displaying discharge instructions for a patient, the user interface comprising:
a first display area configured to display suggested patient education instructions for a patient and a designation indicating the patient education instructions as personal instructions or public instructions, where personal instructions are accessible only to a user inputting the personal instructions;
a second display area configured to display suggested follow-up care instructions for the patient;
a third display area configured to display suggested follow-up appointment instructions for the patient, wherein the suggested follow-up appointment instructions include an automatically generated follow-up appointment; and
a fourth display area configured to display suggested prescriptions instructions for the patient that are automatically suggested along with a suggested dosage based on patient information in an electronic medical record associated with the patient,
wherein the first, second, third, and fourth display areas are presented together and are simultaneously viewable such that each of the patient education instructions, follow-up care instructions, follow-up appointment instructions, and suggested prescriptions instructions for the patient are simultaneously displayed,
wherein the suggested patient education instructions, suggested follow-up care instructions, suggested follow-up appointment instructions, and suggested prescriptions instructions make up a set of discharge instructions based on one or more discharge qualifiers associated with at least one discharge qualification identifier, the set of discharge instructions generated by:
accessing, by a discharge manager in operable communication with a control server, patient data;
accessing a plurality of non-patient-specific discharge qualifiers, each discharge qualifier identifying a diagnosis or symptom description and a demographic trait, and each discharge qualifier associated with one or more suggested discharge instructions based on the discharge qualifier;
applying, by the discharge manager, the plurality of discharge qualifiers to the patient data; and
determining, based on the applying, at least one discharge qualification identifier for the patient, wherein the at least one discharge qualification identifier is generated if the patient data includes one or more discharge qualifiers, applied from the accessed plurality of discharge qualifiers
wherein a single action request from a user for discharge information for the patient triggers automatic determination and display of a set of suggested discharge instructions for the patient.

12. The media of claim 11, wherein the discharge instructions in the first, second, third and fourth display areas may be changed.

13. A system for displaying discharge instructions for a patient, the system comprising:
a receiving component for receiving a single action request from a user for discharge information for the patient that triggers automatic determination of suggested discharge instructions for the patient and display of the determined suggested discharge instructions;
a first accessing component for accessing patient data;
a second accessing component for accessing a plurality of non-patient specific discharge qualifiers, each discharge qualifier identifying a diagnosis or symptom description and a demographic trait, and each discharge qualifier associated with one or more suggested discharge instructions based on the discharge;
a discharge management component to apply the plurality of discharge qualifiers to the patient data;
a determination component that determines at least one discharge qualification identifier for the patient, wherein the at least one discharge qualification identifier is generated if the patient data includes one or more discharge qualifiers, applied from the accessed plurality of discharge qualifiers;
a second determination component to generate a set of discharge instructions for the patient based on the one or more discharge qualifiers associated with the at least one discharge qualification identifier, wherein the set of discharge instructions include:
patient education instructions, follow-up care instructions, follow-up appointment instructions and prescription instructions for the patient;
a displaying component for simultaneously displaying each of the patient education instructions, follow-up care instructions, follow-up appointment instructions including an automatically generated follow-up appointment, and prescription instructions for the patient; and
a modification component for receiving a request to change one or more of the set of discharge instructions for the patient, wherein the request to change is a request to modify, request to add, or a request to delete.

14. The system of claim 13, wherein the patient education instructions comprise identification of one or more patient education references appropriate for the patient at discharge.

15. The system of claim 13, wherein the prescription instructions comprise identification of appropriate prescriptions for the patient at discharge.

16. The system media claim 15, wherein the prescription instructions are determined by utilizing a pharmacy application.

17. The system of claim 13, wherein the follow-up care instructions comprise identification of a time period for follow-up care for the patient after discharge.

18. The system of claim 13, wherein the follow-up appointment instructions comprise one or more scheduled follow-up appointments for a patient after discharge.

19. The system of claim 18, wherein the follow-up appointment instructions are determined using a scheduling application.

20. The system of claim 13, wherein the first accessing component accesses the patient data from the patient's electronic medical record.
